(19) United States
(12) Patent Application Publication
(10) Pub. No.: US 2017/0020720 A1
(43) Pub. Date: Jan. 26, 2017

(54) GARMENT FOR PROVIDING RELIEF FROM MENSTRUAL PAIN

(71) Applicant: Brittany O'Neal, Haslet, TX (US)

(72) Inventor: Brittany O'Neal, Haslet, TX (US)

(21) Appl. No.: 14/809,023

(22) Filed: Jul. 24, 2015

(51) Int. Cl. A61F 7/00; A61H 1/00

(52) U.S. Cl. CPC A61F 7/00; A61H 1/008; A61F 2007/0022; A61F 2007/0023; A61F 2007/0024; A61F 2007/005; A61H 2201/0207; A61H 2201/165

(57) ABSTRACT

A garment for providing relief from menstrual pain, the garment comprising at least one fabric layer conforming to contours of a lower and middle body portion. The fabric layer further comprises a plurality of heating pouches accommodating replaceable heating members, placed on an inner side of the fabric layer. The replaceable heating member is configured to produce heat as a result of an exothermic chemical reaction and transfer the heat to predefined parts of the body for pain relief.

### FIELD OF THE INVENTION

The present invention generally relates to the technical field of garments comprising heating elements and more specifically relates to a girdle type compression garment with heating elements for providing relief from pain including menstrual pain and uterine cramps.

### BACKGROUND

Menstrual cramping, or dysmenorrhea is a medical condition characterized by severe uterine pain during menstrual cycle. Menstrual cramps occurs as a result of contractions of the uterus as it expels ruptured endometrial tissue lining along with clotted blood through the cervix. The underlying pain results from fast and/or hard contraction of uterine muscles leading to severe discomfort around the abdomen, pelvis, lower back, thighs and hips. Menstrual cramping may precede menstruation by several days or may accompany it during the menstrual cycle.

Statistical evidences reveal that menstrual cramping represents one of the major reasons for absence at educational institutions and work place. Traditional methods of treatment for menstrual pain or uterine cramps, include taking an over-the-counter pain killer such as ibuprofen as active ingredient or antispasmodic medications. Another method include intake of calcium, Vitamin D and magnesium supplements. Non-medicinal ways of treatment typically include use of heat around the abdomen, such as a heating pad or by wearing heating garments which utilizes electrical heating element, phase change material, air or fluid flow heat generation and chemical substances to produce heat close to the skin surface of the wearer.

Each aforementioned treatment option has its own set of limitations. Increased intake of ibuprofen may result in adverse drug reactions, as well as associated gastrointestinal disorders, renal problems and other undesirable side effects. Use of heat around the abdomen only offers a temporary relief by briefly masking the pain. Existing heating garments for providing pain relief mostly employ electrical heating elements and the efficiency is limited due to inadequate number and placement of heating pouches. In addition, the garment should be made of expandable, light weight material that fits to different body sizes.

Therefore there still exists a need for a heating garment comprising non-electrical means of heating in order to provide immediate relief from menstrual pain or uterine cramps.

### SUMMARY OF THE INVENTION

The present invention relates to a garment, which includes a lower garment or a compression garment or an undergarment, for providing relief from menstrual pain. The garment comprise at least one fabric layer conforming to contours of a lower body portion and a plurality of heating pouches comprising replaceable heating members, placed on an inner side of the fabric layer. The replaceable heating members are configured to produce optimum heat for pain relief, as a result of at least one chemical reaction and transfer the heat to predefined parts of the body.

In an embodiment, the garment of the present invention comprises a girdle consisting multiple heating pouches placed on the inner side of the fabric such as spandex. The heating pouches comprise replaceable heating members configured to produce optimum heat for pain relief. The heating pouches are placed in such a way to provide heat to the surface of predefined parts of body such as thighs, hips, lower back, lower abdomen and pelvic regions. The heat is produced as a result of chemical reaction and provides a soothing and comforting effect thus providing relief from menstrual pain.

In an embodiment, the heating member comprises iron which reacts with air to form iron oxide and the reaction generates optimum heat that is required for pain relief. In an embodiment, the heating member is configured to generate heat sufficient enough for pain relief without burning the skin. The heating members are interchangeable and replaceable. The garment of the present invention can be worn as an undergarment beneath any type of clothing, thus concealed from external view.

In an embodiment, the garment of the present invention may comprise a high waist girdle made of elastic fabric which provides a slim appearance to the wearer and also controls abdomen bloating associated with the menstrual period. In another embodiment, the heating members can be removed from the pouches and the garment can then be used as a regular slimming garment.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1A is a front view of the garment showing the placement of heating pouches.

FIG. 1B is a sectional view of the region 1B-1B shown in FIG. 1A.

FIG. 2 is a rear view of the garment showing the placement of heating pouches.

FIG. 3 is a perspective view of the garment.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

The present invention relates to a garment for providing relief from menstrual pain during menstrual period and also can be used as a slimming garment during normal times. The garment may comprise a compression garment embedded with non-electrical heating elements or heating members configured to produce an optimum range of heat, which is transferred to specific regions of body which are prone to pain during menstrual cycle, thereby providing immediate relief from menstrual pain and uterine or abdominal cramps.

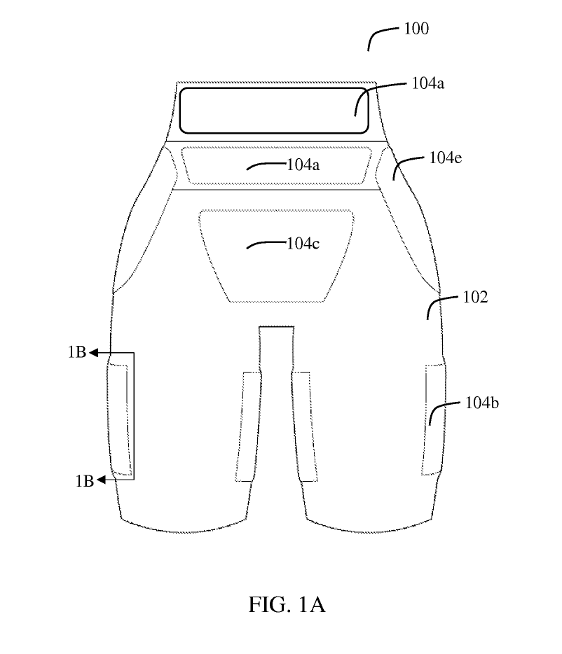

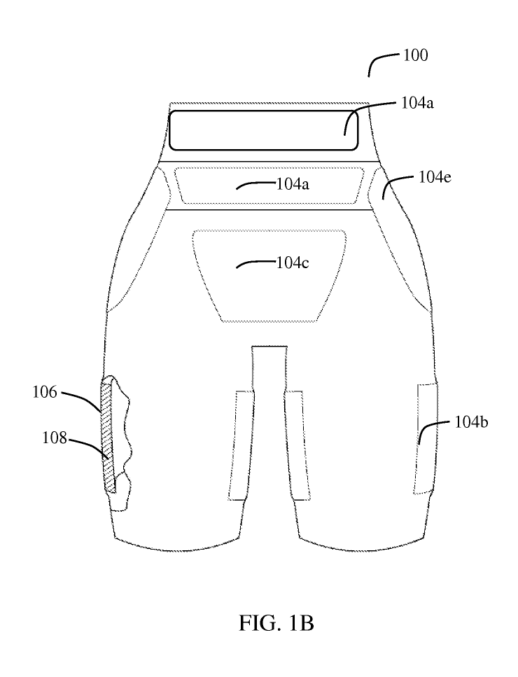

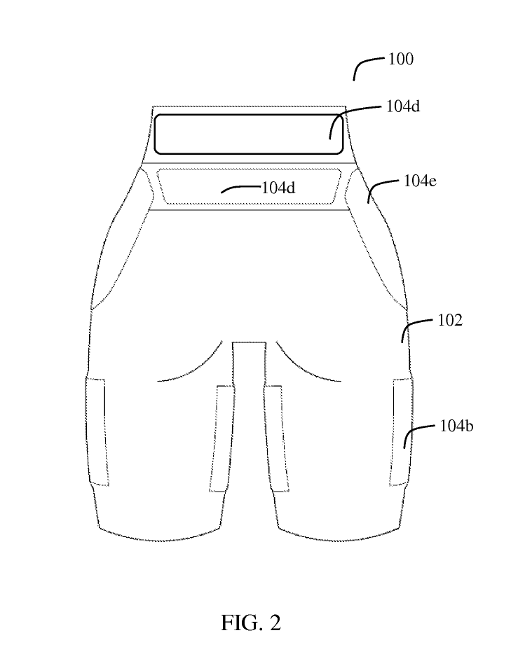

Referring to FIG. 1A and FIG. 2, which illustrates a front view and a rear view of the garment 100 comprising at least one fabric layer 102 conforming to contours of a lower body portion of a female user. In an embodiment, the garment 100 may comprise two or more fabric layers conforming to the shape of the lower body region. A plurality of heating pouches 104a-104e comprising replaceable heating members, are placed on an inner side of the fabric layer 102. The replaceable heating members are configured to produce optimum heat for pain relief, as a result of at least one chemical reaction and transfer the heat to predefined parts of the body.

In an embodiment, the heating pouches 104a-104e are placed within the garment such as to provide an optimum heat for pain relief at regions such as lower abdomen 104a, thigh 104b, pelvis 104c, lower back 104d, and hip 104e. The heating pouches can also be placed in additional lower body regions that are prone to pain associated with menstruation.

FIG. 1B illustrates a sectional view of the region 1B-1B of the garment 100 shown in FIG. 1A. The heating pouches 104a-104e may comprise a heating member 106 consisting a packet filled with chemical substance 108 which is configured to react with air or water to produce heat. In an embodiment, the chemical substance 108 comprise iron which is configured to react with air to produce iron oxide and heat. In another embodiment, the chemical substance may comprise iron and sodium chloride, which accelerates the reaction. Alternative chemical substances that are capable of exothermic chemical reaction and safe on the skin, can also be used as reactants.

Heat released from the chemical reaction provides a comforting warmth to the body parts adjacent to the reaction site. In an embodiment, the gaseous reaction products provides an optimum range of heat required for pain relief from uterine and/or abdominal cramps. For example, the heating member 106 produces a temperature sufficient enough to provide pain relief without causing any skin burn or heat related injuries to the body of the wearer. The heating members 106 are interchangeable and replaceable.

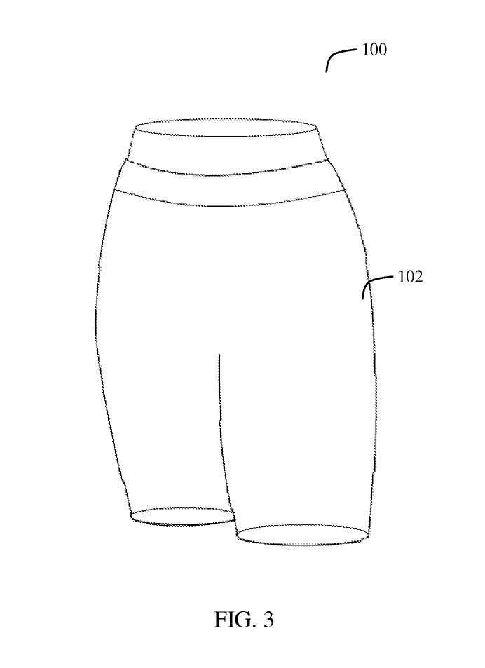

FIG. 3 illustrates a perspective view of the garment 100, which is made up of one or more layers of fabric 102 which is elastic and conforms to the contours of the body. For example, the fabric 102 may comprise natural elastic fibers or synthetic elastic fibers such as spandex or elastane. The fabric 102 is durable, strong, and washable besides offering high elasticity. The garment 100 may comprise a compression garment or an undergarment.

In an embodiment, the garment 100 may comprise a high waist girdle made of elastic fabric which provides a slim appearance to the wearer and also decreases the appearance of abdomen bloating associated with the menstrual period. In another embodiment, the heating members can be removed from the pouches and the garment can then be used as a regular slimming garment or shape wear or bodysuit such as corset, girdle, and the like.

In another embodiment, the garment 100 can be customized according to user requirement. The user can chose to place or remove one or more heating members 106 based on requirement for pain relief at specific regions of the body. For example, if the user is suffering from severe abdominal pain, then the user can place heating members 106 only in the pouches at lower abdomen 104a and pelvic region 104c.

Additional advantages and modifications will also readily occur to those skilled in the art. Therefore, the invention in its broader aspects is not limited to the specific details and representative devices, shown and described herein. Accordingly, various modifications may be made without departing from the spirit and scope of the general inventive concept as defined by the appended claims and their equivalents.

What is claimed is:

1. A garment for providing relief from pain, the garment comprising:
at least one fabric layer conforming to contours of a lower body portion; and
a plurality of heating pouches comprising replaceable heating members, placed on an inner side of the fabric layer,
wherein the replaceable heating member is configured to produce heat as a result of chemical reaction and transfer the heat to predefined parts of the body for pain relief.

2. The garment of claim 1, wherein the pain comprise menstrual pain.

3. The garment of claim 1, wherein the fabric layer comprise spandex.

4. The garment of claim 1, wherein the replaceable heating member comprise iron.

5. The garment of claim 1, wherein the replaceable heating member comprise a chemical substance capable of exothermic reaction upon contact with air.

6. The garment of claim 1, wherein the heating pouches are placed at regions configured to be in contact with the skin at thigh, hip, lower abdomen, lower back and pelvis.
